(19) United States
(12) Patent Application Publication
(10) Pub. No.: US 2004/0121026 A1
(43) Pub. Date: Jun. 24, 2004

(54) Non-toxic composition for the control of epiphytic weeds

(76) Inventor: Jeffrey Lee Kaplan, Fort Lauderdale, FL (US)

Correspondence Address: JEFFREY KAPLAN, POB 11106, Ft. Lauderdale, FL, 33339, US

(21) Appl. No.: 10/326,938

(22) Filed: Dec. 23, 2002

(51) Int. Cl. A61K 33/04; A61K 35/78; A61K 33/00

(52) U.S. Cl. 424/705; 424/717; 424/776

(57) ABSTRACT

A non-toxic formulation to selectively control and kill undesirable epiphytic weeds by applying active ingredients in an effective amount of sodium bicarbonate, potassium salts of fatty acids, sulfur, sesame oil and water.

### BACKGROUND

1. Field Of Invention

This invention relates to novel compositions comprising mixtures of sodium bicarbonate and non-toxic ingredients and to a method of controlling undesired epiphytic species by the application of an effective amount of one of the compositions that comprise the mixture, alone or the composition comprising the mixtures.

2. Discussion Of Prior Art

The genus Tillandsia which belongs to the Bromeliaceae includes almost 400 species distributed from the south of the United States Of America to the northern part of Patagonia in Argentina. The population of these epiphytic species such as Tillandsia Recurvata and Tillandsia Aeranthos on trees and shrubs in the southernmost western and eastern regions of the United States has increased dramatically to epidemic proportions, causing severe deleterious effects on their hosts.

Epiphytes have been generically known as symbionic air plants and for three decades have been considered harmless to their hosts. Typically, epiphytic plants are not rooted in soil. Instead, they live above ground level on the stems and branches of other plants and trees. It's roots penetrate the subsurface of the phorophyte (tree) and they girdle the hosts branches Once these epiphytes are established on a tree or other host they derive their nutrients by using their trichomes (leaf hairs) to scavenge water and nutrients from airborne particles and leachates in precipitation and from moisture in the air. They also obtain minerals from organic matter that has accumulated on the surface of the host plant on which they are growing.

For seven years the present inventor has studied this extensive growth and their infestation patterns throughout the United States and South America. This inventor has studied the deleterious effects of these epiphytes and has concluded that they should be redefined as “epiphytic weeds”. These epiphytic weeds should be categorized as “nutritional pirates” whereby they steal the life giving nutrients that are needed by their host thus causing the decline and eventual death of its host.

These epiphytic weeds have a great multiplication capacity owing to the numerous plume seeds that allow them to fix onto the wrinkled surfaces of trees and branches. A severe infestation of the growth of these epiphytic weeds on a host causes significant shade which in turn causes light deprivation to the hosts foilage. In addition these epiphytes may be allelopathic, secreting a toxic substance and or growth inhibitor which is responsible for leaf abscission Once this nutritional pirate takes over it's host canopy, the weight of the epiphytic load usually breaks branches. Resulting from their canopy position on the hosts, epiphytes such as the Tillandsia specie individuals cannot be easily controlled by cutting or pulling without damaging the tree bark, thus selective “Non-Toxic” compositions/formulations are needed and are in great demand. The use of selective toxic herbicides to control various weeds is well established in the art. Typical herbicides that are commercially available are 2,4-dichlorophenoxyacetic acid Copper Sulfate, Cutrine, Thiocarbamtes, Butylate, CDEC, Diallate, EPTC, Kocide, Glyphosate, MCPP, Diquat, Triclopyr, CMA, Sethoxydim and many others. The majority of commercially available herbicides that control weeds are limited to soil application where they are readily absorbed from the soil by roots and translocated via the xylem to the foliage where they inhibit oxygen evolution in photosynthesis.

Typical weed control herbicidal formulations are set forth in the U.S. Pat. No. 5,420,098 to Hodogaya Chemical Co. Ltd (1995), in U.S. Pat. No. 5,668,089 to Zeneca Ltd (1997), in U.S. Pat. No. 5,696,024 to American Cyanamid Co. (1997) and in U.S. Pat. No. 5,981,434 to Jeff Kaplan (1999). Unfortunately, these patent formulations as well as others have many disadvantages such as toxicity, flammability, and are all, except the cited Kaplan's patent, primarily used in the soil. The toxic level in the cited Kaplan formulation is very low compared to the other cited patents but nevertheless is still slightly toxic. This slight toxicity may affect the vigor and health of adjacent un-infested plants and trees exposed through spraying in crowded urban areas. It has been discovered by the inventor that there is no cited prior art relating to the control of epiphytes using completely biodegradable “Non-Toxic” formulations/compositions. Thus, a need exists for a safe and reliable biodegradable composition/formulation that is specifically designed to control the unique problems associated with epiphytic weeds and their respective physiology.

### QBJECTS AND ADVANTAGES

Accordingly, several objects and advantages of my invention are:

a) to provide a novel non-toxic formulation designed specifically to control the growth of epiphytic weeds.

b) to provide a novel nontoxic formulation that will be absorbed through the trichomes of the epiphyte.

c) to provide a novel non-toxic formulation that can be absorbed by the epiphyte without affecting the host species and causing phytotoxic effects on the terminal buds and leaves of the host plant.

d) to provide a novel non-toxic formulation that will not harm adjacent plants, trees and greenery during the application process to the epiphytic infested hosts.

e) to provide a novel non-toxic formulation that is completely safe for humans and wildlife when used at the recommended doses.

f) to provide a novel non-toxic biodegradable formulation.

g) to provide a novel non-toxic formulation that is easily dispersed onto the epiphytes and is inexpensive.

### DETAILED DESCRIPTION OF THE INVENTION

In the present invention, the foregoing difficulties are obviated in that there is provided a low cost, easily dispersed non-toxic and biodegradable formulation consisting of commercially available ingredients In accordance with the invention, the formulation is initially comprised of: sodium bicarbonate mixed with various biodegradable ingredients such as potassium salts of fatty acids, sulfur, and sesame oil. These ingredients can be applied in eight different formulations. (1) Sodium Bicarbonate and water. (2) Sodium Bicarbonate, sulfur and water. (3) Sodium Bicarbonate, potassium salts of fatty acids and water. (4) Sodium Bicarbonate, potassium salts of fatty acids, sulfur and water. (5) Sodium Bicarbonate, Sesame Oil and water (6) Sodium Bicarbonate, Sesame Oil, potassium salts of fatty acids and water. (7) Sodium Bicarbonate, sulfur, sesame oil, potassium salts of fatty acids and water. (8) Sodium Bicarbonate, sulfur, sesame oil and water. The formulation is sprayed onto the foliage of the host and to the epiphytic weeds. The doses are chosen by a dose response assay and is familiar to those skilled in the art. The formulated mixtures can be dispersed using conventional methods such as knapsack, power and bottle sprayers as well as aircraft sprayers. These mixtures produce a synergistic action and have an increase in the effectiveness of the treatment. During June 1998 to September 1998 in the area of Fort Lauderdale Fla. the inventor tested these formulations on five different host species which were actively growing and were infested with epiphytes of the tillandsia species All the selected formulations, alone or in combination controlled and killed the epiphytes without causing phytotoxicity to their host plant nor did it affect or injure any plant or greenery in close proximity.

### CONCLUSION, RAMIFICATION AND SCOPE OF THE INVENTION

Accordingly, the reader will see that a selectively formulated non-toxic biodegradable composition of the invention provides that:

It will specifically control and kill the growth of epiphytic weeds.

It will be absorbed through the trichomes of the epiphytes.

It will be absorbed by the epiphyte without affecting the host species and causing phytotoxic effects on the terminal buds and leaves of the host plant.

It will not harm adjacent plants, trees and greenery during the application process to the epiphytic infested hosts.

It will provide a non-toxic formulation that is completely safe for humans and wildlife when used at the recommended doses.

It will provide a non-toxic biodegradable formulation.

It will provide a non-toxic formulation that is easily dispersed onto the epiphytes and is inexpensive.

Those skilled in the art will have no difficulty in determining suitable proportions of the above compositions to be used. The invention has been described as applied to preferred embodiments and it will be understood that various substitutions and changes may be effected without departing from the spirit and scope of the novel concepts and principals of this invention.

I claim:

1. A non toxic biodegradable formulation consisting essentially of specific ingredients or a combination of specific ingredients mixed with water selected from a group consisting of sodium bicarbonate, potassium salts of fatty acids, sulfur, and sesame oil.

2. A non-toxic biodegradable formulation as in claim 1 consisting of sodium bicarbonate and water.

3. A non-toxic biodegradable formulation as in claim 1 consisting of sodium bicarbonate, potassium salts of fatty acids and water.

4. A non-toxic biodegradable formulation as in claim 1 consisting of sodium bicarbonate, sulfur and water.

5. A non-toxic biodegradable formulation as in claim 1 consisting of sodium bicarbonate, sesame oil and water.

6. A non-toxic biodegradable formulation as in claim 1 consisting of sodium bicarbonate, potassium salts of fatty acids, sulfur and water.

7. A non-toxic biodegradable formulation as in claim 1 consisting of sodium bicarbonate, potassium salts of fatty acids, sulfur, sesame oil and water.

8. A non-toxic biodegradable formulation as in claim 1 consisting of sodium bicarbonate, sulfur, sesame oil and water.

9. A non-toxic biodegradable formulation as in claim 1 consisting of sodium bicarbonate, potassium salts of fatty acids, sesame oil and water.

10. A method or controlling and eradicating the species tillandisa recurvats and other epiphytic weeds which comprises applying the composition of any one of claims 1-9 to the branches and canopies of the infected hosts.
